(19) United States
(12) Patent Application Publication
(10) Pub. No.: US 2009/0068288 A1
(43) Pub. Date: Mar. 12, 2009

(54) Topical antiseptic veterinary solution

(76) Inventor: Wayne Kruger, Charlotte, MI (US)

Correspondence Address: PAULEY PETERSEN & ERICKSON, 2800 WEST HIGGINS ROAD, SUITE 365, HOFFMAN ESTATES, IL, 60195, US

(21) Appl. No.: 11/899,735

(22) Filed: Sep. 7, 2007

(51) Int. Cl. A61K 33/36; A61P 43/00

(52) U.S. Cl. 424/670; 424/671

(57) ABSTRACT

This invention relates to a veterinary antiseptic solution having three sources of iodine including elemental iodine, iodide salt and iodophor. The solution also includes at least one solvent such as alcohol, glycol or water. The solution is below the threshold concentration for elemental iodine of the List I controlled substances. The elemental iodine offers a rapid kill for bacteria. The iodophor offers lasting antibacterial properties. The iodide salt solubilizes elemental iodine and stabilizes the solution. The antiseptic solution forms a waterproof bandage with active antimicrobial properties when the alcohol dries.

### BACKGROUND OF THE INVENTION

1. Field of the Invention

This invention relates to a topical antiseptic veterinary solution having three sources of iodine. The solution results in a soft waterproof antibacterial bandage.

2. Description of Related Art

It is known to use various compounds or solutions to reduce infection of skin wounds on domestic animals. Iodine is recognized to be effective as a veterinary antiseptic including strong iodine tinctures previously commonly available in 7.0 percent weight per volume iodine, USP (United States Pharmacopeia) concentration.

The escalating use of elemental iodine for illicit purposes including narcotics production resulted in a change in U.S. law. Elemental iodine solutions exceeding concentrations of 2.2 percent weight per volume are presently listed as a federally controlled substance (List I) requiring proper registration. Many farmers performing animal husbandry practices, such as dipping a newborn calf's navel to prevent infection, will not have access to List I chemicals since most over-the-counter retailers will not register with the DEA (U.S. Drug Enforcement Administration).

There is a need for an effective antiseptic solution that contains elemental iodine below the threshold concentration of the List I chemicals and is effective as the conventional higher concentration iodine tinctures.

Conventional antiseptic solutions have a good initial antibacterial effect but lack an active antibacterial residue to kill future microbes. Other conventional antiseptic solutions have an active antibacterial residue without good initial antimicrobial effect. Conventional antiseptic solutions are comparatively thin (low viscosity) and do not form a bandage or more desirably a waterproof bandage having lasting antibacterial properties when dried. There is a need for a waterproof bandage forming antiseptic solution.

Conventional iodine antiseptic solutions contain a higher concentration of elemental iodine and alcohol that may be painful and irritating to the skin. The conventional antiseptic solutions also may cause scar tissue and may slow the healing process. There is a need for a milder antiseptic solution with the claimed features.

### SUMMARY OF THE INVENTION

Accordingly, it is one object of this invention to provide a topical antiseptic veterinary solution addressing one or more of the needs discussed above.

A topical antiseptic veterinary solution according to a preferred embodiment of this invention comprises three sources of iodine and at least one solvent. The three sources of iodine include elemental iodine, iodide salt and iodophor. These three sources of iodine combine to form a solution with quick antimicrobial activity from the elemental iodine, lasting antimicrobial activity from the iodophor and stable chemistry from the iodide salt.

A significant benefit of the solution of this invention is an effective antiseptic solution with an elemental iodine content below the DEA List I threshold of 2.2 percent weight per volume. Therefore, the solution is not a controlled substance and registration is not required.

According to a preferred embodiment of this invention the titratable iodine does not exceed about 4.0 percent weight per volume including elemental iodine and titratable iodine from the iodophor.

The solvent may include water, glycol and/or alcohol. Desirably, upon evaporation of the alcohol, a soft waterproof bandage with active antiseptic properties remains. A preferred glycol is propylene glycol which also acts as a humectant. A preferred alcohol is isopropyl alcohol which may be about 99 percent pure. The antiseptic solution typically includes a lower alcohol content than a conventional iodine tincture which reduces irritation or burning sensation when applied. Desirably, purified and/or sterilized water is added to the solution in a suitable amount.

The antiseptic solution of this invention may be applied to treat scrapes, cuts, incisions, insect bites, abrasions, minor burns, minor bruises, and/or lesions, for example. Animals treated with the antiseptic solution include, without limitation, dogs, cats, cattle, sheep, horses, swine and/or any other suitable domesticated or wild animals.

Elemental iodine, typically in a crystal form, dissolves in the solution with the aid of the alcohol and/or an aqueous solution of the iodide salt, for example. Potassium iodide is a preferred iodide salt and povidone iodine is a preferred iodophor. A preferred polymer in the iodophor is polyvinylpyrrolidone.

Antiseptic solutions are effective against bacteria, germs, viruses, fungi, spores, yeasts, protozoa, eukaryotic cells, prokaryotic cell, gram-positive cells, and/or gram-negative cells. The term effective refers to reducing a significant amount of a potential pathogen, desirably at least below a potentially harmful or disease causing level.

The components of the solution can be varied to produce a solution of desired properties. According to a preferred embodiment, the antiseptic solution includes: about 0.1 percent weight per volume to about 2.2 percent weight per volume elemental iodine; about 1.0 percent weight per volume to about 7.0 percent weight per volume potassium iodide; about 1.0 percent weight per volume to about 8.0 percent weight per volume povidone iodine; about 2.0 percent weight per volume to about 15 percent weight per volume propylene glycol; about 15.0 percent weight per volume to about 30.0 percent weight per volume isopropyl alcohol; and about 50.0 percent weight per volume to about 70.0 percent weight per volume water.

One or more oxidizing agents may be added to the solution to further enhance the antibacterial properties of the solution.

Other objects and advantages will be apparent to those skilled in the art from the following detailed description taken in conjunction with the appended claims.

### DETAILED DESCRIPTION OF THE PREFERRED EMBODIMENTS

This invention relates to a veterinary antiseptic solution applied to, for example, scrapes, cuts, incisions, insect bites, abrasions, minor burns, minor bruises, lesions, sores, blisters, rashes, hives, superficial lacerations and/or any other suitable surface irritation or wound opening. The term antiseptic broadly includes substances applied to living tissue to reduce, prevent or at least partially inhibit growth of potentially harmful contaminants, such as causing infection, sepsis, illness, putrefaction and/or any other disease.

Possible contaminants may include, without limitation, germs, microbes, bacteria, viruses, fungi, yeasts, spores, protozoa, pathogens, eukaryotic cells, prokaryotic cell, gram-positive cells, gram-negative cells and/or any other undesirable DNA containing substance or transmissible agent. Desirably, antiseptic solutions or compounds are at least partial germicides and include antimicrobial properties.

The solution of this invention may also function or act as a disinfectant. Disinfectants destroy microorganisms on non-living objects and/or surfaces. Sterilization refers to at least partially reducing reproduction and/or growth capabilities of a treated subject. Sanitize refers to making healthy, hygienic and/or clean, such as killing at least 99.9 percent of bacteria. The solution of this invention may also cleanse the skin of animals as a prophylactic or preventative measure, for example, before surgery.

The term veterinary broadly refers to the practice of the science and/or art of prevention, alleviation and/or cure of injury and/or disease in animals, such as domestic animals. Domestic animals may include, without limitation, dogs, cats, cattle, sheep, horses, swine, lizards, reptiles, poultry, fowl, fish, primates and/or any other animals at least partially tamed or cared for by human beings. According to a preferred embodiment of this invention, the animals are mammals.

Antiseptic solutions typically are applied topically or to the surface of the skin, scalp, dermal layer, hair-covered area, shaved hair-covered areas, fur, coats, mucous membranes, epithelial cells and/or any other suitable portion of a patient, such as a side of a horse bit by a number of mosquitoes.

The term solution broadly refers to a combination, mixture and/or admixture of ingredients having at least one liquid component. Solutions typically include at least one solvent in greater quantity or volume than a solute. Typical solvents include water, alcohols, aldehydes, ketones, monomers and/or any other suitable solubilizing molecule or carrier compound. Typical solutes include elemental materials, compounds, salts, complexes and/or any other suitable substance at least partially dissolved or suspended in or by the solvent.

Those skilled in the art and guided by the teachings herein appreciate that solutions may encompass other materials beyond classical solution chemistry, such as, for example, emulsions, extracts, tinctures, suspensions, gels, pastes, colloids and/or any other suitable mixture form. Aqueous solutions use water as a primary solvent. Tinctures use alcohol as at least a portion of the primary solvent.

Other agents or ingredients may be added to the solution, such as to affect physical properties, including, but not limited to, color, viscosity, density and/or any other suitable characteristic.

According to a preferred embodiment of this invention, the antiseptic solution includes three sources of iodine: elemental iodine, iodide salt and iodophor. The three sources or types of iodine each provide individual effectiveness as well as a combined synergistic benefit.

The first source of iodine, elemental iodine or crude iodine, is number 53 on the periodic table and is a very reactive halogen element, group VII. Elemental iodine is typically water insoluble but soluble in alcohol and soluble in aqueous solutions of iodide ions, such as potassium iodide and water. Elemental iodine is so reactive that it is not found in its crystalline diatomic state in nature. Elemental iodine is high reactively toward organic compounds, rendering it suitable as an antiseptic agent.

On Aug. 1, 2007 the DEA (United States Drug Enforcement Administration) moved elemental iodine to a List I chemical requiring registration for any product containing greater than 2.2 percent weight per volume elemental iodine. Conventional strong iodine tinctures, such as 7.0 percent weight per volume, are thus a controlled substance requiring proper registration and handling. The rules and regulations were published in the Jul. 2, 2007 Federal Register (Volume 72, Number 126) and modifies 21 CFR Parts 1309 and 1310, particularly 1310.12 for exempt chemical mixtures. Other forms of iodine, such as iodophors and iodide salts have not been diverted for illicit uses (methamphetamine production). Iodophors and iodide salts remain exempted from the List I chemicals since they contain no elemental iodine used for illegal purposes.

The antiseptic solution according to a preferred embodiment of this invention may contain elemental iodine in any desired amount. According to a preferred embodiment of this invention and remaining below the threshold for a List I classification, the solution includes elemental iodine in no greater amount than about 2.2 percent weight per volume of the solution. According to other preferred embodiments the solution includes about 0.1 percent weight per volume to about 2.2 percent weight per volume elemental iodine and more preferably about 2.1 percent weight per volume.

A second source of iodine, iodide salts, broadly may include iodine and any suitable ionic bond forming element or compound, such as alkali metals, group I, including potassium and sodium. Iodide salts typically are soluble in water as an anion, such as I⁻. According to a preferred embodiment of this invention, the iodide salt comprises potassium iodide.

Iodide salts typically exhibit little antibacterial properties but promote the complexing and/or coordination of elemental iodine, I₂ to I₃⁻ which increases the solubility and the stability of elemental iodine in alcohol, water and combinations thereof. Sometimes dissolving elemental iodine in a solution of water and iodide salt may be referred to as Lugol's iodine.

The antiseptic solution according to a preferred embodiment of this invention may include iodide salt in any desired amount. According to a preferred embodiment of this invention, the iodide salt is about 1.0 percent weight per volume to about 7.0 percent weight per volume of the solution, more preferably about 1.0 percent weight per volume to about 5.0 percent weight per volume and most preferably about 2.0 percent weight per volume.

The third source of iodine, iodophors, broadly includes compounds with surface active agents to act as surfactants, carrier agents or solubilizing agents for iodine, such as nonoxynol or polymers. Iodophors typically enhance bactericidal or germicidal activity of iodine while reducing vapor pressure and odor of iodine. Iodophors reduce and/or eliminate staining while allowing wide dilution with water, for example.

According to a preferred embodiment of this invention, the iodophor includes povidone iodine or sometimes referred to as PVP-Iodine which is soluble in water, ethanol and propanol. Before incorporation into the solution, the iodophor may take a powder form. After incorporation into the solution, the concentration of the iodophor may directly increase the viscosity of the solution. Polyvinylpyrrolidone is a desired polymer for an iodophor.

The antiseptic solution according to a preferred embodiment of this invention may include iodophor in any desired amount. According to a preferred embodiment of this invention, the solution includes about 1.0 percent weight per volume to about 8.0 percent weight per volume iodophor, more preferably about 1.0 percent weight per volume to about 5.0 percent weight per volume, and most preferably about 3.0 percent weight per volume.

According to a preferred embodiment of this invention, the antiseptic solution may include a glycol. The glycol may include ethylene glycol, propylene glycol and/or any other suitable diol compound. Typically, the glycol acts as a humectant to promote the retention of moisture, for example. The humectant characteristics serve to prevent a drying effect, such as when a relatively low molecular weight alcohol is part of the solution. The glycol may further increase the viscosity of the solution.

The antiseptic solution according to a preferred embodiment of this invention may include glycol in any desired amount. According to a preferred embodiment of this invention, the glycol is about 2.0 percent weight per volume to about 15.0 percent weight per volume of the solution, more preferably about 1.0 percent weight per volume to about 10.0 percent weight per volume and most preferably about 6.0 percent weight per volume.

According to a preferred embodiment of this invention, the antiseptic solution may include an alcohol. Alcohols include any suitable hydroxyl containing compound, such as methanol, ethanol, propanol and/or isopropanol. Desirably, the alcohol is fully or at least partially miscible with water. As described above, the elemental iodine dissolves in the alcohol. Desirably, the alcohol itself may further promote and/or impart antimicrobial properties to the solution. Denatured alcohols unfit for human consumption may be used in the solution preparation.

The antiseptic solution according to a preferred embodiment of this invention may include alcohol in any desired amount. According to a preferred embodiment of this invention, the alcohol is about 5.0 percent weight per volume to about 50.0 percent weight per volume of the solution, more preferably about 15.0 to about 30.0 percent weight per volume and most preferably about 23.7 percent weight per volume. Desirably, the formulation minimizes alcohol content for the purpose of cost and/or to reduce stinging, such as when applying the solution to open sores.

Any suitable purity of alcohol may be used, such as, for example, about 99 percent pure isopropyl alcohol. Distillation, extraction, salting out, desiccation and/or any other suitable azeotrope breaking equipment or technique may be used to achieve the desired alcohol separation or purity. When water is also added to the antiseptic solution advantageously lower purity alcohols may be used, such as for cost reduction.

According to a preferred embodiment of this invention, the solution includes water, such as a solvent. The water may be purified, such as by distillation, softening, chlorination, sterilization, irradiation, ion exchange, reverse osmosis and/or any other suitable impurity reducing process.

The antiseptic solution according to a preferred embodiment of this invention may include water in any desired amount. According to a preferred embodiment of this invention, the water is about 20.0 percent weight per volume to about 85.0 percent weight per volume, more preferably about 50.0 percent weight per volume to about 70.0 percent weight per volume of water and most preferably about 63.2 percent weight per volume.

As mentioned above, the antiseptic solution of the combined forms of iodine has synergistic benefits, for example the iodide salt helps to solubilize and/or stabilize the elemental iodine in the water. The elemental iodine is quicker to reduce bacteria than the iodophor alone. The iodophor remains antibacterially active on the treated surface longer than just the elemental iodine. Another benefit is less stinging or irritation since a lower concentration of elemental iodine is in the solution as allowed by the iodophor.

Additionally, the combination of the three sources of iodine allows an effective antiseptic solution while remaining below DEA List I threshold levels for elemental iodine.

According to a preferred embodiment of this invention, the antiseptic solution titrates to less than 4.0 percent weight per volume iodine (elemental iodine and titratable iodine from the iodophor). This 4.0 percent weight per volume titratable iodine material may be formulated below the threshold levels for elemental iodine by keeping the elemental iodine less than 2.2 percent weight per volume.

The water in combination with the glycol as solvents allow a reduced amount of alcohol needed for sufficient solution stability, thus there is less stinging or burning from application of the antiseptic solution, such as to bleeding skin.

According to a preferred embodiment of this invention, the solution includes the three sources of iodine, glycol, water and alcohol. The alcohol readily dries, evaporates or flashes when applied, for example to warm animal skin. The elemental iodine rapidly kills the bacteria, the alcohol evaporates forming a film or bandage of the iodophor (active iodines), water (drying), glycol and iodide salt.

Desirably and unexpectedly, a soft supple bandage formed from the residue or remainder of the solution is waterproof. Thus, an in-situ waterproof antibacterial bandage results. The bandage possesses adhesive properties evidenced by samples of the antiseptic solution contacting and drying on Teflon® coated laboratory equipment. The resultant material is not easily removed. This solution may be applied, for example, to a cow's ear when placing a plastic identification tag while out in the pasture.

Varying the relative amounts of the components of the antiseptic solution varies the resultant properties of the bandage formed when the alcohol evaporates. For example, the elemental iodine may be reduced and the iodophor and glycol increased to yield a softer, milder and more easily washed off bandage with less promotion of scar tissue. Such a mild formulation may be suitable for sensitive areas, including such as a teat dipping product and use on teats or teat areas.

The solution may be supplied in any suitable or convenient packaging, such as glass or plastic bottles in 1 pint, 1 quart and 1 gallon volumes, for example. Alternately, the container may encompass a pouch such as Mylar®.

Desirably, the viscosity of the solution allows use of a spray or an aerosol dispensing mechanism, such as a squeeze trigger disposed on a neck of a bottle. Those skilled in the art and guided by the teachings herein readily appreciate that the solution may be adapted to various other delivery forms, such as by changing a carrier or solvent. It is possible to produce salves, balms, liniments, gels, ointments, creams, compresses, wraps, gauzes, sponges and/or any other suitable shape or form having the antiseptic properties of the three sources of iodine.

The solution may be manufactured in any suitable batch or continuous method. According to a preferred embodiment a method of production includes the following amounts.

TABLE 1

| | | |
|---|---|---|
| Weight | Weight per volume | |
| (kilograms) | (percent) | Component |

| | | | |
|---|---|---|---|
| 151 | 2.0 | | Iodide Salt |
| 1,794 | 23.7 | | Alcohol |
| 454 | 6.0 | | Glycol |
| 159 | 2.1 | | Elemental Iodine |
| 227 | 3.0 | | Iodophor |
| q.s. | 7570 | liters | Water |

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

What is claimed is:

1. A topical antiseptic veterinary solution comprising:
elemental iodine;
potassium iodide;
povidone iodine; and
glycol.

2. The topical antiseptic veterinary solution of claim 1 wherein the glycol comprises propylene glycol.

3. The topical antiseptic veterinary solution of claim 1 wherein the solution is effective against at least one of the group consisting of bacteria, spores, viruses, fungi, yeasts, protozoa, germs, eukaryotic cells, prokaryotic cell, gram-positive cells, and gram-negative cells.

4. The topical antiseptic veterinary solution of claim 1 further comprising at least one of alcohol and water.

5. The topical antiseptic veterinary solution of claim 4 wherein the alcohol comprises at least one of ethanol and isopropyl alcohol.

6. The topical antiseptic veterinary solution of claim 4 wherein a film forms with active antiseptic properties and remains after evaporation of the alcohol.

7. The topical antiseptic veterinary solution of claim 1 wherein:
the elemental iodine is no greater than about 2.2 percent weight per volume of the solution;
the potassium iodide is about 1.0 percent weight per volume to about 5.0 percent weight per volume of the solution;
the povidone iodine is about 1.0 percent weight per volume to about 5.0 percent weight per volume of the solution; and
the glycol is about 1.0 percent weight per volume to about 10 percent weight per volume of the solution.

8. A topical antiseptic veterinary solution comprising:
elemental iodine;
iodide salt; and
iodophor;
wherein the solution titrates to less than 4.0 percent weight per volume iodine.

9. The topical antiseptic veterinary solution of claim 8 wherein the iodophor comprises polyvinylpyrrolidone.

10. The topical antiseptic veterinary solution of claim 8 wherein the iodide salt comprises potassium iodide.

11. The topical antiseptic veterinary solution of claim 8 wherein:
the solution is applied to at least one of the group consisting of scrapes, cuts, incisions, insect bites, abrasions, minor burns, minor bruises, and lesions.

12. The topical antiseptic veterinary solution of claim 8 wherein:
the solution is applied to at least one of the group consisting of dogs, cats, cattle, sheep, horses, and swine.

13. The topical antiseptic veterinary solution of claim 8 wherein:
the elemental iodine is no greater than about 2.2 percent weight per volume of the solution;
the potassium iodide is about 1.0 percent weight per volume to about 5.0 percent weight per volume of the solution; and
the povidone iodine is about 1.0 percent weight per volume to about 5.0 percent weight per volume of the solution.

14. The topical antiseptic veterinary solution of claim 13 further comprising:
isopropyl alcohol; and
water.

15. The topical antiseptic veterinary solution of claim 14 wherein:
the isopropyl alcohol is about 5.0 percent weight per volume to about 50.0 percent weight per volume of the solution; and
the water is about 20.0 percent weight per volume to about 85.0 percent weight per volume of the solution.

16. A topical antiseptic veterinary solution comprising:
about 0.1 percent weight per volume to about 2.2 percent weight per volume elemental iodine;
about 1.0 percent weight per volume to about 7.0 percent weight per volume potassium iodide;
about 1.0 percent weight per volume to about 8.0 percent weight per volume povidone iodine;
about 2.0 percent weight per volume to about 15.0 percent weight per volume propylene glycol;
about 15.0 percent weight per volume to about 30.0 percent weight per volume isopropyl alcohol; and
about 50.0 percent weight per volume to about 70.0 percent weight per volume of water.

17. The topical antiseptic veterinary solution of claim 16 wherein:
the elemental iodine is about 2.1 percent weight per volume;
the potassium iodide is about 2.0 percent weight per volume;
the povidone iodine is about 3.0 percent weight per volume;
the propylene glycol is about 6.0 percent weight per volume;
the isopropyl alcohol is about 23.7 percent weight per volume; and
the water is about 63.2 percent weight per volume.

18. The topical antiseptic veterinary solution of claim 16 wherein:
the isopropyl alcohol is about 99 percent pure.

19. The topical antiseptic veterinary solution of claim 16 wherein:
the water is purified.
